(19) United States
(12) Patent Application Publication
(10) Pub. No.: US 2026/0204173 A1
(43) Pub. Date: Jul. 16, 2026

(54) EXTENDED REALITY SYSTEM FOR UNIPORTAL THORACOSCOPIC SURGERY

(71) Applicants: National Taiwan University, Taipei City (TW); QUANTA COMPUTER INC., Taoyuan City (TW)

(72) Inventors: Yi-Ping Hung, Taipei City (TW); Jin-Shing Chen, Taipei City (TW); Yang-Sheng Chen, Taipei City (TW); Xu-Heng Chiang, Taipei City (TW); Hsien-Yuan Hsieh, Taipei City (TW); Yi-Ching Lee, Taipei City (TW)

(21) Appl. No.: 19/410,060

(22) Filed: Dec. 5, 2025

Related U.S. Application Data
(60) Provisional application No. 63/744,403, filed on Jan. 13, 2025.

(51) Int. Cl. G09B 9/00; A61B 1/00; A61B 1/313; A61B 34/00; A61B 34/10; A61B 90/00; G06F 3/01; G06T 19/00; G06T 19/20

(52) U.S. Cl. CPC G09B 9/00; A61B 1/00048; A61B 1/3132; A61B 34/10; A61B 34/25; A61B 90/37; A61B 90/39; G06F 3/011; G06T 19/006; G06T 19/20; A61B 2090/365; A61B 2090/372; A61B 2090/3937; G06T 2200/24; G06T 2210/41; G06T 2219/2016

(57) ABSTRACT

An extended reality system for uniportal thoracoscopic surgery is disclosed. The extended reality system involves a head-mounted display device, communicated with a first handheld controller; a visual auxiliary marker, disposed on a preset object and communicated with the head-mounted display device; a handheld controller scaffold, disposed at a predetermined position and communicated with the head-mounted display device; and an external body stabilization device, disposed on a patient and communicated with the head-mounted display device, the external body stabilization device is configured to ensure that the patient's posture is consistent when obtaining a medical image and performing a surgical simulation using the head-mounted display device, thereby assisting a three-dimensional model to coincide with the real objects in the real environment under the perspective condition.

### PRIOR ART

In recent years, virtual reality and extended reality technologies have gradually matured, and their applications in medical-related fields have become increasingly diverse and extensive, including medical education, telemedicine, and medical treatment simulation training. Through the virtual world or platform created by relevant technologies, users can transcend the limitations of time or space at will, improving the implementation and risks of medical treatment while reducing risks to patients.

In the aspect of the application of virtual reality or extended reality in preoperative planning of surgical operations, this broad category of products can be applied in surgical fields of different subspecialties, allowing users to review two-dimensional medical images or three-dimensional reconstructions before surgery to enhance their understanding of surgical anatomy. Alternatively, a simulation platform may be provided for users to simulate surgical procedures on digital twin organs before surgery. Alternatively, it can provide a communication and discussion platform to allow multiple users to conduct detailed preoperative planning in a virtual reality or augmented reality environment.

In terms of integrating virtual reality or extended reality with preoperative planning of surgical operations, some companies have launched related systems that provide immersive three-dimensional visual experience and the ability to review medical images. For example, the SurgicalAR extended reality system sold by Medivis overlays reconstructed two-dimensional medical images onto the patient's body using optically realistic three-dimensional graphics, allowing users to assess surgical anatomy before surgery. However, the actual endoscopic field of view of thoracoscopic surgery cannot be simulated during the operation. Therefore, the planning of uniportal thoracoscopic surgery still has problems such as incomplete opening planning and inability to optimize the surgical field of view.

Therefore, based on the aforementioned requirements of uniportal thoracoscopic surgery, an innovative guided extended reality system is needed. It integrates an immersive 3D virtual reality viewing environment, extended reality technology for inspecting digital twin organs and patients, and simulates the dynamic field of view in the thoracoscopic camera to achieve the effectiveness of uniportal thoracoscopic surgery planning and simulation, thereby solving the problems of the conventional technology mentioned above.

### BACKGROUND OF THE INVENTION

The present invention relates to the technical field of extended reality, in particular to an extended reality system that utilizes a see-through head-mounted display to inspect, evaluate, and measure three-dimensional digital twin organs reconstructed from medical images, and then a positioning technology is then used to superimpose the twin organs on the patient's body in a real environment to assist in surgical planning and simulation of single-port thoracoscopic surgery.

### SUMMARY OF THE INVENTION

A primary objective of the present invention is to provide an extended reality system for uniportal thoracoscopic surgery. The hardware configuration includes a see-through head-mounted display device, a visual auxiliary marker for positioning, a handheld controller scaffold for positioning, and an external body stabilization device for positioning. The extended reality system provides immersive virtual reality and extended reality that interacts with the real world, allowing users to perform surgical planning, digital twin organ reference, and thoracoscopic simulation effects during single-port thoracoscopic surgery.

In order to achieve the aforementioned objective, the present invention provides an extended reality system for uniportal thoracoscopic surgery. The extended reality system includes a head-mounted display device, communicated with a first handheld controller; a visual auxiliary marker, disposed on a preset object and communicated with the head-mounted display device, the visual auxiliary marker is configured to provide a first position coordinate to the head-mounted display device; a handheld controller scaffold, disposed at a predetermined position and communicated with the head-mounted display device, the handheld controller scaffold is configured to calibrate a second position coordinate of a real environment and a three-dimensional model of an extended reality in the head-mounted display device to assist in aligning the three-dimensional model with real objects in the real environment under a perspective condition; and an external body stabilization device, arranged on a patient and communicated with the head-mounted display device, the external body stabilization device is configured to ensure that a patient's posture is consistent when obtaining a medical image and performing a surgical simulation using the head-mounted display device, thereby assisting the three-dimensional model to coincide with the real objects in the real environment under the perspective condition.

In a preferred embodiment of the present invention, the extended reality system further comprises a user interface, communicated with the first handheld controller, the user interface is configured to allow a user to use the first handheld controller to move the three-dimensional model in all directions and rotate the three-dimensional model in an immersive viewing environment, and to measure a vertical distance and a curvilinear distance of each structure in the three-dimensional model.

In a preferred embodiment of the present invention, the handheld controller scaffold is communicated with the head-mounted display device through a second handheld controller.

In a preferred embodiment of the present invention, the head-mounted display device utilizes a see-through display technology to overlay and inspect the patient in the real environment, and a user freely sets an opening for a thoracoscope using the first handheld controller in the extended reality, and simulates a dynamic field of view in a lens of the thoracoscope by moving the first handheld controller.

In a preferred embodiment of the present invention, the handheld controller scaffold comprises a rigid material, and the rigid material is plastic.

In a preferred embodiment of the present invention, the external body stabilization device comprises a rigid material or a fillable material, the rigid material is plastic and the fillable material is a foaming agent.

In a preferred embodiment of the present invention, the first position coordinate of the visual auxiliary marker is a two-dimensional barcode that can be recognized by the head-mounted display.

In a preferred embodiment of the present invention, the preset object at least includes a patient's body, each real object in the real environment, or the external body stabilization device.

In a preferred embodiment of the present invention, the head-mounted display device is used for the extended reality, the first handheld controller is used to inspect, measure and interact with a three-dimensional digital twin organ, thereby providing an immersive visual feedback to a user.

In a preferred embodiment of the present invention, the user interface is further configured to allow the user to use the first handheld controller to open, eliminate, or render transparent a specific structure in the immersive viewing environment through a select menu for easy inspecting.

The extended reality system for uniportal thoracoscopic surgery of the present invention utilizes extended reality to present three-dimensional visualization of twin organs and a position overlap technology and a virtual thoracoscopic field of view simulation allow users to experiment with thoracoscopic opening views from different perspectives. This provides an easy-to-use and practical tool to enhance the user's understanding of three-dimensional surgical anatomy, optimize the performance of uniportal thoracoscopic surgery, and reduce the surgical risks that may accompany inappropriate openings.

### BRIEF DESCRIPTION OF DRAWINGS

Aspects of the present invention are best understood from the following detailed description when read with the accompanying figures. It is noted that, in accordance with the standard practice in the industry, various features are not drawn to scale. In fact, the dimensions of the various features may be increased or reduced for clarity of discussion.

FIG. 1 is a schematic diagram of an extended reality system for uniportal thoracoscopic surgery of the present invention.

FIG. 2 is a block diagram of the extended reality system for uniportal thoracoscopic surgery of the present invention.

FIG. 3 is a schematic diagram of the use process of the extended reality system for uniportal thoracoscopic surgery of the present invention.

FIG. 4 is a schematic diagram of the application results of the extended reality system for uniportal thoracoscopic surgery of the present invention.

### DETAILED DESCRIPTION OF THE INVENTION

It will be appreciated that, although specific embodiments of the present invention are described herein for purposes of illustration, various modifications may be made without departing from the spirit and scope of the present invention.

In the following description, certain specific details are set forth in order to provide a thorough understanding of various aspects of the disclosed subject matter. However, the disclosed subject matter may be practiced without these specific details. In some instances, well-known structures and methods of power delivery comprising embodiments of the subject matter disclosed herein have not been described in detail to avoid obscuring the descriptions of other aspects of the present invention.

Unless the context requires otherwise, throughout the specification and claims that follow, the word “comprise,” “have,” “include,” and variations thereof, such as “comprises,” “comprising,” “having,” “including” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present invention.

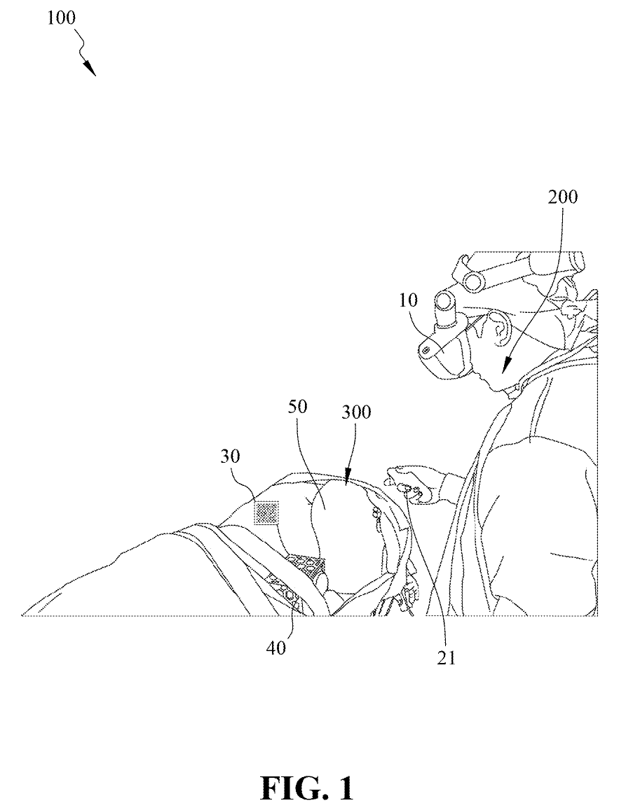

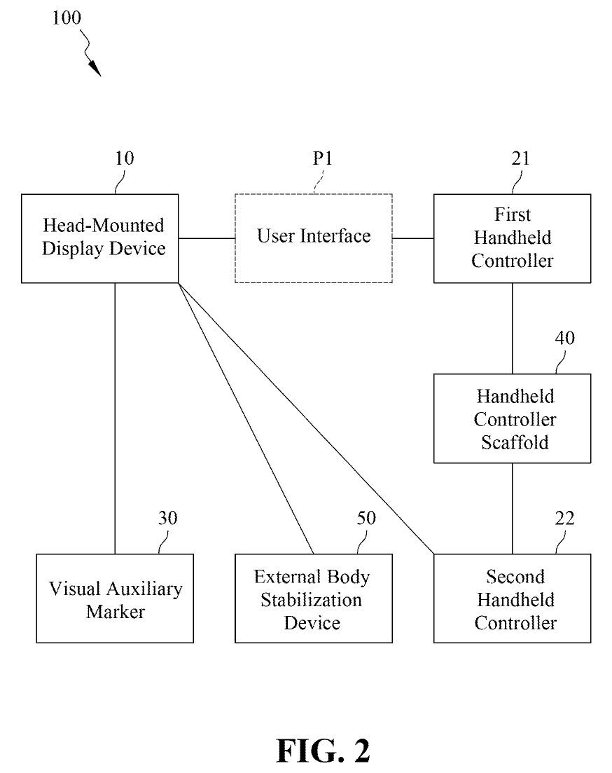

FIG. 1 is a schematic diagram of an extended reality system for uniportal thoracoscopic surgery of the present invention. FIG. 2 is a block diagram of the extended reality system for uniportal thoracoscopic surgery of the present invention.

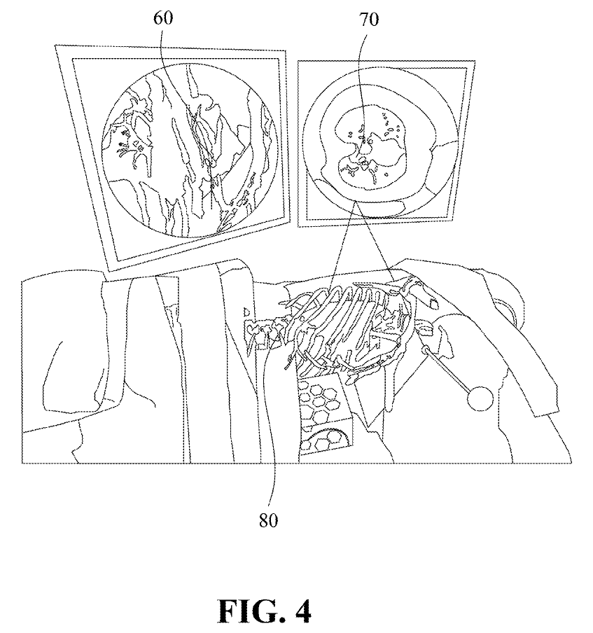

Please refer to FIG. 1 and FIG. 2, the extended reality system 100 for uniportal thoracoscopic surgery of the present invention is used to provide an immersive three-dimensional virtual reality viewing environment, or utilize perspective technology to overlay and reference the patient in the real environment for viewing, allowing a user 200 (e.g., a physician/doctor, but not limited thereto) to inspect a three-dimensional model positioning and simulate a dynamic field of view of the thoracoscopic camera (e.g., the simulated thoracoscopic viewing window 60 shown in FIG. 4) under predetermined functions and interfaces, thereby achieving the purpose of surgical planning and simulation. In some embodiments, the extended reality system 100 for uniportal thoracoscopic surgery of the present invention may include a head-mounted display device 10, a visual auxiliary marker 30, a handheld controller scaffold 40, and an external body stabilization device 50.

The head-mounted display device 10 may be communicated with a first handheld controller 21. In some embodiments, the head-mounted display device 10 is configured for extended reality, and the first handheld controller 21 (or, a gesture controller) may be configured to inspect, measure, and interact with a three-dimensional digital twin organs 80 (as shown in FIG. 4), thereby providing immersive visual feedback to the user 200.

In some embodiments, the head-mounted display device 10 may utilize see-through display technology to overlay and reference the patient 300 in the real environment. Furthermore, the user 200 may freely set a thoracoscopic opening using the first handheld controller 21 in the extended reality system 100 of the present invention, and simulate the dynamic field of view in the lens of a thoracoscopic (not shown) by moving the first handheld controller 21 (for example, the simulated thoracoscopic viewing window 60 shown in FIG. 4). In a real surgical scenario, after the patient enters an anesthetized state, the user 200 may utilize the extended reality system 100 of the present invention to plan and simulate an uniportal thoracoscopic surgery, and actually mark an actual thoracoscopic opening on the patient 300 under fluoroscopic conditions, thereby enabling the planned thoracoscopic setup and surgical process to be implemented after removing the extended reality system 100 of the present invention.

Please refer to FIG. 1 and FIG. 2 again, the visual auxiliary marker 30 may be disposed on a preset object and communicated with the head-mounted display device 10. The visual auxiliary marker 30 may be configured to provide a first position coordinate to the head-mounted display device 10. In some embodiments, the preset object at least includes a body of the patient 300, various real objects in the real environment, or the external body stabilization device 50. In some embodiments, the first position coordinate of the visual auxiliary marker 30 is a two-dimensional barcode capable for being recognized by the head-mounted display 10. The visual auxiliary marker 30 may be used as a positioning auxiliary object to transmit the coordinate information (i.e., the first position coordinate) of the real environment to the head-mounted display device 10 under perspective conditions. The visual auxiliary marker 30 may be fixed on a patient's body in a real environment (i.e., the body of the patient 300), a real scene object (i.e., each real object in the real environment), or the external body stabilization device 50. It includes a material that may be displayed in a specific medical image, thereby maintaining the relative position of each anatomical structure during the process of converting a two-dimensional sequence image (e.g., the two-dimensional medical image viewing window 70 shown in FIG. 4) into a three-dimensional model.

In some embodiments, the visual auxiliary marker 30 also includes a mark (including a two-dimensional barcode capable for being recognized by the head-mounted display device 10, but not limited thereto) whose coordinates (i.e., the first position coordinates) may be positioned, thereby calibrating the real environment and a second position coordinate of a three-dimensional model in extended reality, and further assisting the three-dimensional model to overlap with objects in the real environment under perspective conditions. In addition, the visual auxiliary marker 30 may also support a dynamic positioning coordinate calibration, thereby achieving dynamic tracking and superposition of the three-dimensional model in a situation where the user 200 and the patient 300 have relative displacement.

The handheld controller scaffold 40 may be disposed at a predetermined position and communicated with the head-mounted display device 10. The handheld controller scaffold 40 may be configured to calibrate the second position coordinates of the real environment and the three-dimensional model of an extended reality in the head-mounted display device 10 to assist in aligning the three-dimensional model with real objects in the real environment under perspective conditions.

In some embodiments, the handheld controller scaffold 40 may be used as a positioning auxiliary object. The handheld controller holder 40 may comprise a rigid material (including plastic, but not limited thereto) and may fix another handheld controller (e.g., the second handheld controller 22) therein. The position information (e.g., position coordinates) of the second handheld controller 22 is thereby transmitted to the head-mounted display device 10 to calibrate the real environment and the position coordinates of the three-dimensional model in the extended reality, so as to help the three-dimensional model to coincide with the real objects in the real environment under perspective conditions.

The external body stabilization device 50 may be configured to arrange on the patient 300 and communicated with the head-mounted display device 10. The external body stabilization device 50 may be configured to ensure that the patient 300 maintains a consistent posture when acquiring a medical image and performing a surgical simulation using the head-mounted display device 10, thereby assisting in aligning the three-dimensional model with real objects in the real environment under perspective conditions.

In some embodiments, the external body stabilization device 50 may be used as a positioning auxiliary object. The external body stabilization device 50 may comprise a rigid material (including plastic, but not limited thereto) or a fillable material (including foaming agent, but not limited thereto). The posture of the patient 300 when obtaining medical images and performing surgical simulation using the extended reality system 100 of the present invention is made consistent, thereby assisting in aligning the three-dimensional model with real objects in the real environment under perspective conditions.

The extended reality system 100 of the present invention may further include a user interface P1 communicating with the first handheld controller 21. The user interface P1 may be configured to allow the user 200 to use the first handheld controller 21 to move the three-dimensional model in various directions and rotate the three-dimensional model in an immersive viewing environment, and measure a vertical distance and a curvilinear distance of each structure in the three-dimensional model. In some embodiments, the user interface P1 may be further configured to allow the user 200 to utilize the first handheld controller 21 to open, clear, or render transparent a specific structure through a select menu in the immersive viewing environment for easier inspecting. Therefore, when the see-through display is superimposed on the patient 300 in the real environment, the user 200 may move and rotate the three-dimensional model in all directions, move the virtual section and inspect the corresponding two-dimensional medical image (for example, the two-dimensional medical image viewing window 70 shown in FIG. 4), or open, eliminate, or render transparent a specific structure through a menu-based user interface for easier inspecting. For example, utilizing in uniportal thoracoscopic sublobectomy, first, the user 200 may fix the visual auxiliary marker 30 at a predetermined location on the patient 300 (e.g., the spine, the lower edge of the ribs, or an anatomical landmark near the surgical site), and then use the external body stabilization device 50 to reproduce and fix the patient's posture (e.g., embracing the device with both hands, lying on the side with the surgical site facing upward). Under the see-through display condition, the superimposing function is triggered by the first handheld controller 21 to calculate a transformation matrix between the real space (i.e., real environment) coordinates of the visual auxiliary marker 30 identified by the head-mounted display device 10 and the relative coordinates of the three-dimensional model to achieve the alignment of the three-dimensional model and the patient's position in the real environment. For another example, in some embodiments, another handheld controller (e.g., a second handheld controller 22) may be fixed to a predetermined position (e.g., the lower edge of the sternum) on the patient 300 using a handheld controller scaffold 40, and the second handheld controller 22 transmits the real space (i.e., the real environment) coordinates to the head-mounted display device 10 to achieve the conversion operation of the real space (i.e., the real environment) coordinates and the alignment of the three-dimensional model.

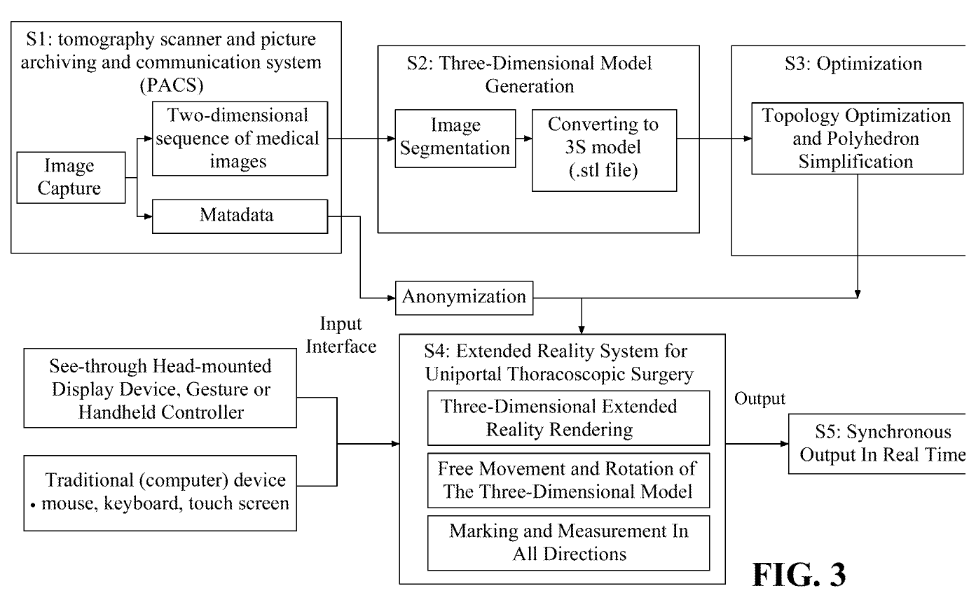

FIG. 3 is a schematic diagram of the use process of the extended reality system for uniportal thoracoscopic surgery of the present invention.

Please refer to FIG. 3, steps S1 to S3 are performed before the operation, while steps S4 and S5 are performed during or after the operation.

In step S1, a two-dimensional sequence of medical images (Including fine-section (meaning the interval between sections is less than 1 mm) (e.g., the two-dimensional medical image viewing window 70 shown in FIG. 4) CT images and MRI images, but not limited thereto) and a matadata of the patient 300 are first obtained through a tomography scanner and a picture archiving and communication system (PACS). In step S2, an image is segmented to subdivide the image into a plurality of image sub-regions corresponding to different organs or anatomical structures, and each image sub-region is converted into a three-dimensional model. In step S3, appropriate objects are optimized. In step S4, a three-dimensional image and attribute information of the three-dimensional digital twin organ 80 are imported into the extended reality environment and inspected using the head-mounted display device 10, wherein the used input interface may include the head-mounted display device 10, the first handheld controller 21, and a traditional (computer) device (such as a mouse, keyboard, touch screen, etc.). In step S5, the three-dimensional extended reality is outputted in real time as a rendered image. For example, when used in single-port thoracoscopic sublobectomy, in step S1, a chest CT image and the metadata of the patient 300 are obtained by a CT scanner; in step S2, it is subdivided into sub-regions such as skin, bones, pulmonary circulation arteries and veins, systemic circulation arteries and veins, trachea and bronchi, and tumors and converted into a three-dimensional model; in step S3, topology optimization is performed on high-polyhedron objects with multiple faces such as bones and lung lobes; in step S4, the three-dimensional digital twin organ 80 and attribute information of the above models are imported into the extended reality environment; and in step S5, the three-dimensional extended reality image is outputted in real time via the head-mounted display device 10.

In some embodiments, in step S2, the image segmentation technology first utilizes a segmentation model based on a neural network or threshold detection to distinguish structures including skin, bones, pulmonary circulation arteries and veins, systemic circulation arteries and veins, trachea and bronchi, tumors, etc. (but not limited thereto) on a two-dimensional sequence of medical images. Next, the user 200 may optimize the boundaries and branches of specific structures (e.g., the distal ends of blood vessels and bronchi) according to the requirements of the surgical planning, or divide specific substructures into separate blocks and convert each block into a three-dimensional model to achieve the pre-operative planning and thoracoscopic simulation effect that the user 200 considers to be optimal.

In some embodiments, in step S3, the object optimization technique may reduce the number of faces of the three-dimensional model generated in step S2 by approximately 50% through repeated vertex pruning and secondary edge collapse while maintaining the geometric integrity of each structure; then, Laplace smoothing is used to smooth the surface, and weighted normal vectors are used to optimize the lighting and shadow effects. The optimized objects may be imported into the extended reality environment and inspected using the head-mounted display device 10.

In some embodiments, the step S5 further includes an image stream. The image of the head-mounted display device 10 may be transmitted to a local or cloud server via real-time network transmission technology (e.g., WebRTC), and then streamed to a browsing device of other viewers (including head-mounted displays, mobile devices, but not limited thereto).

In addition, the extended reality system 100 of the present invention may be configured to communicate to a cloud recording device (not shown) and automatically record and upload the user status and the user's usage history to the cloud recording device for reference by the user or medical care personnel, thereby realizing the cloud recording function.

To clearly illustrate the specific effects that can be achieved by the extended reality system 100 of the present invention, reference may be made to the exemplary embodiment shown in FIG. 1. Before performing an uniportal thoracoscopic surgery, after the patient 300 enters an anesthetized state, the user 200 may utilize the extended reality system 100 of the present invention to plan the uniportal thoracoscopic surgery, and the extended reality system 100 of the present invention sequentially simulates the field of view under different thoracoscope opening positions and the operability during the surgery.

After determining the most suitable thoracoscopic surgical opening position, the user 200 can actually mark the thoracoscopic opening on the patient 300 under fluoroscopic conditions, thereby realizing the planned thoracoscopic setup and surgical process after removing the extended reality system 100 of the present invention.

Therefore, in the extended reality function created by the extended reality system 100 of the present invention, the user 200 can use three-dimensional vision to try out different perspectives of the thoracoscopic opening without performing invasive treatment on the patient 300, so as to optimize the performance of the uniportal thoracoscopic surgery and reduce the surgical risks that may be associated with inappropriate openings, including prolonged operation time and damage to normal tissues and organs, but not limited thereto.

The extended reality system 100 of the present invention may adjust the parameters (e.g., transparency, etc.) of various parts of the three-dimensional model according to the visual needs of the user 200, thereby optimizing the visual experience of the user 200 and the integrity of the surgical planning. For example, when the user 200 wants to inspect the location of a tumor inside the lung, the user may adjust the lung to a semi-transparent display through the graphical interface so that the specific location of the tumor inside may be inspected.

In summary, the extended reality system 100 for uniportal thoracoscopic surgery of the present invention is disclosed. The hardware configuration includes a see-through head-mounted display device, a visual auxiliary marker for positioning, a handheld controller scaffold for positioning, and an external body stabilization device for positioning. The extended reality system provides immersive virtual reality and extended reality that interacts with the real world, allowing users to perform surgical planning, digital twin organ reference, and thoracoscopic simulation effects during single-port thoracoscopic surgery.

The above descriptions are only used to explain the preferred embodiments of the present invention, and are not intended to limit the present invention in any form. Therefore, any modifications or changes made to the present invention under the same inventive spirit should still be included in the scope of protection intended by the present invention.

1. An extended reality system for uniportal thoracoscopic surgery, comprising:
a head-mounted display device, communicated with a first handheld controller;
a visual auxiliary marker, disposed on a preset object and communicated with the head-mounted display device, the visual auxiliary marker is configured to provide a first position coordinate to the head-mounted display device;
a handheld controller scaffold, disposed at a predetermined position and communicated with the head-mounted display device, the handheld controller scaffold is configured to calibrate a second position coordinate of a real environment and a three-dimensional model of an extended reality (i.e., Virtual Reality, VR) in the head-mounted display device to assist in aligning the three-dimensional model with real objects in the real environment under a perspective condition; and
an external body stabilization device, arranged on a patient and communicated with the head-mounted display device, the external body stabilization device is configured to ensure that a patient's posture is consistent when obtaining a medical image and performing a surgical simulation using the head-mounted display device, thereby assisting the three-dimensional model to coincide with the real objects in the real environment under the perspective condition.

2. The extended reality system for uniportal thoracoscopic surgery according to claim 1, further comprising a user interface, communicated with the first handheld controller, the user interface is configured to allow a user to use the first handheld controller to move the three-dimensional model in all directions and rotate the three-dimensional model in an immersive viewing environment, and to measure a vertical distance and a curvilinear distance of each structure in the three-dimensional model.

3. The extended reality system for uniportal thoracoscopic surgery according to claim 1, wherein the handheld controller scaffold is communicated with the head-mounted display device through a second handheld controller.

4. The extended reality system for uniportal thoracoscopic surgery according to claim 1, wherein the head-mounted display device utilizes a see-through display technology to overlay and inspect the patient in the real environment, and a user freely sets an opening for a thoracoscope using the first handheld controller in the extended reality, and simulates a dynamic field of view in the lens of the thoracoscope by moving the first handheld controller.

5. The extended reality system for uniportal thoracoscopic surgery according to claim 1, wherein the handheld controller scaffold comprises a rigid material, and the rigid material is plastic.

6. The extended reality system for uniportal thoracoscopic surgery according to claim 1, wherein the external body stabilization device comprises a rigid material or a fillable material, the rigid material is plastic and the fillable material is a foaming agent.

7. The extended reality system for uniportal thoracoscopic surgery according to claim 1, wherein the first position coordinate of the visual auxiliary marker is a two-dimensional barcode that can be recognized by the head-mounted display.

8. The extended reality system for uniportal thoracoscopic surgery according to claim 1, wherein the preset object at least includes a patient's body, each real object in the real environment, or the external body stabilization device.

9. The extended reality system for uniportal thoracoscopic surgery according to claim 1, wherein the head-mounted display device is used for the extended reality, the first handheld controller is used to inspect, measure and interact with a three-dimensional digital twin organ, thereby providing an immersive visual feedback to a user.

10. The extended reality system for uniportal thoracoscopic surgery according to claim 2, wherein the user interface is further configured to allow the user to use the first handheld controller to open, eliminate, or render transparent a specific structure in the immersive viewing environment through a select menu for easy inspecting.
